(19) United States
(12) Patent Application Publication
(10) Pub. No.: US 2022/0291137 A1
(43) Pub. Date: Sep. 15, 2022

(54) SURFACE INSPECTION APPARATUS

(71) Applicant: FUJIFILM Business Innovation Corp., Tokyo (JP)

(72) Inventors: Takashi HIRAMATSU, Kanagawa (JP); Kaito TASAKI, Kanagawa (JP); Kiyofumi AIKAWA, Kanagawa (JP); Miho UNO, Kanagawa (JP); Hiroko ONUKI, Kanagawa (JP); Hirokazu ICHIKAWA, Kanagawa (JP); Yoshitaka KUWADA, Kanagawa (JP)

(73) Assignee: FUJIFILM Business Innovation Corp., Tokyo (JP)

(21) Appl. No.: 17/381,653

(22) Filed: Jul. 21, 2021

(30) Foreign Application Priority Data
Mar. 10, 2021 (JP) 2021-038682

(51) Int. Cl. G01N 21/88; G01N 33/44; G01N 21/95

(52) U.S. Cl. CPC G01N 21/8851; G01N 33/442; G01N 21/95; G01N 2021/8887

(57) ABSTRACT

A surface inspection apparatus includes an imaging device that images a portion of an object to be inspected, a first light source that is included in multiple light sources that illuminate the portion and that is configured such that a light component that is included in light emitted from the first light source and that is reflected by specular reflection from the portion to be inspected is a principal light component that is incident on the imaging device, and a second light source that is included in the multiple light sources and that is disposed opposite the first light source with an optical axis of the imaging device interposed therebetween such that a light component that is reflected by diffuse reflection from the portion to be inspected is a principal light component that is incident on the imaging device.

### CROSS-REFERENCE TO RELATED APPLICATIONS

This application is based on and claims priority under 35 USC 119 from Japanese Patent Application No. 2021-038682 filed Mar. 10, 2021.

### BACKGROUND

### (i) Technical Field

The present disclosure relates to a surface inspection apparatus.

### (ii) Related Art

For various products, components (referred to below as “molded components”) composed of molded synthetic resin are used. In some cases, defects that are visually observable appear on surfaces of the molded components. Examples of these kinds of defects include a “sink mark” that is a depression that is unintentionally formed and a “weld line” that is formed on a portion to which melted resin joins. Also, in some cases where unevenness is intentionally formed on a surface by texturing processing, there is a difference in texture from supposed texture. The texture changes due to a composite factor of color, gloss, and unevenness.

Defects that are visually observable are visually inspected.

Japanese Patent No. 5765152 is an example of related art.

### SUMMARY

There are various proposed methods for a device that inspects the surface state of an object to be inspected. However, these need special optical systems, and there are no devices that enable the defects and the texture to be inspected at low costs.

Aspects of non-limiting embodiments of the present disclosure relate to an inspection of the defects and the texture at lower costs than those in the case where the special optical systems are used.

Aspects of certain non-limiting embodiments of the present disclosure address the above advantages and/or other advantages not described above. However, aspects of the non-limiting embodiments are not required to address the advantages described above, and aspects of the non-limiting embodiments of the present disclosure may not address advantages described above.

According to an aspect of the present disclosure, there is provided a surface inspection apparatus including an imaging device that images a portion of an object to be inspected, a first light source that is included in a plurality of light sources that illuminate the portion, the first light source being configured such that a light component that is included in light emitted from the first light source and that is reflected by specular reflection from the portion to be inspected is a principal light component that is incident on the imaging device, and a second light source that is included in the plurality of light sources and that is disposed opposite the first light source with an optical axis of the imaging device interposed therebetween such that a light component that is reflected by diffuse reflection from the portion to be inspected is a principal light component that is incident on the imaging device.

### BRIEF DESCRIPTION OF THE DRAWINGS

Exemplary embodiments of the present disclosure will be described in detail based on the following figures, wherein:

FIG. 1 illustrates an example of the use of a surface inspection apparatus that is supposed according to a first exemplary embodiment;

FIG. 2A and FIG. 2B illustrate examples of defects that appear on a surface to be inspected where FIG. 2A illustrates sink marks by way of example, and FIG. 2B illustrates a weld line by way of example;

FIG. 3 illustrates an example of the hardware configuration of the surface inspection apparatus that is used according to the first exemplary embodiment;

FIG. 4 illustrates an example of the structure of an optical system in the surface inspection apparatus according to the first exemplary embodiment;

FIG. 5 is a flowchart illustrating an example of the inspection operation of the surface inspection apparatus;

FIG. 6A, FIG. 6B, FIG. 6C, and FIG. 6D illustrate the principle of the inspection of the surface inspection apparatus according to the first exemplary embodiment where FIG. 6A illustrates an image C by way of example, FIG. 6B illustrates a section of a depressed defect that is formed on the surface to be inspected, FIG. 6C illustrates a luminance profile SA of an image A and a luminance profile SB of an image B, and FIG. D illustrates a luminance profile SA-SB related to the image C and the luminance profile SA of the image A;

FIG. 7A and FIG. 7B illustrate examples of the image C that is displayed according to a second exemplary embodiment where FIG. 7A illustrates, by way of example, the image C that is acquired by imaging an inspection object and that is displayed on a display as it is, and FIG. 7B illustrates, by way of example, the image C that is acquired by imaging the inspection object and that is displayed with an indicator superposed thereon;

FIG. 8A and FIG. 8B illustrate examples of the image C that is displayed according to a third exemplary embodiment where FIG. 8A illustrates a position at which the indicator is attached, and FIG. 8B illustrates an example of the image C that is acquired by imaging the inspection object and that is displayed;

FIG. 9 illustrates the arrangement of an optical system in a surface inspection apparatus according to a fourth exemplary embodiment; and

FIG. 10 illustrates an example of the use of a surface inspection apparatus that is supposed according to a fifth exemplary embodiment.

### DETAILED DESCRIPTION

Exemplary embodiments of the present disclosure will hereinafter be described with reference to the drawings.

### First Exemplary Embodiment

### Example of Use of Surface Inspection Apparatus

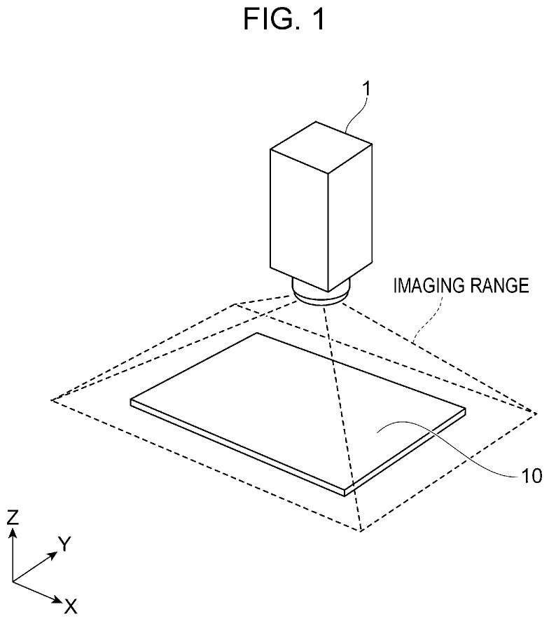

FIG. 1 illustrates an example of the use of a surface inspection apparatus 1 that is supposed according to a first exemplary embodiment.

The surface inspection apparatus 1 that is used according to the first exemplary embodiment is a so-called area camera. A range that the surface inspection apparatus 1 images (referred to below as an “imaging range”) is defined by a plane.

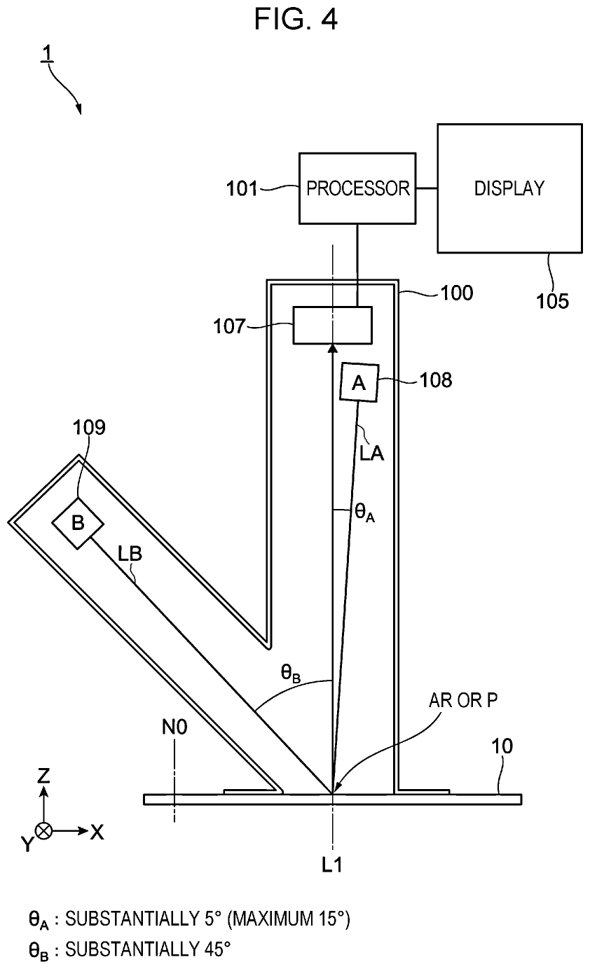

In FIG. 1, an illustration of a light-shielding frame 100 (see FIG. 4) that shields the imaging range from incident natural light is omitted. The light-shielding frame 100 is configured by using a material or a member that does not allow the natural light to pass through.

The light-shielding frame 100 is also used for position adjustment between the surface inspection apparatus 1 and an inspection object 10 in addition to natural light shielding. Example of the position adjustment described herein include position adjustment for the imaging range, position adjustment between a surface of the inspection object 10 and light sources 108 and 109 (see FIG. 4), and position adjustment between the surface of the inspection object 10 and a camera 107 (see FIG. 4).

The light-shielding frame 100 configures a part of the surface inspection apparatus 1. The light-shielding frame 100 and the surface inspection apparatus 1 may have a single body, or the light-shielding frame 100 may be attachable to and detachable from the housing of the surface inspection apparatus 1.

In FIG. 1, the imaging range covers the entire object 10 to be inspected (also referred to below as the “inspection object”). However, the imaging range may cover only a portion to which attention is paid regarding the inspection object 10. According to the present exemplary embodiment, a molded component is supposed as the inspection object 10.

An inspection with the area camera is conducted with the surface inspection apparatus 1 and the inspection object 10 being at rest. In other words, the surface of the inspection object 10 is inspected in a state in which the surface inspection apparatus 1 and the inspection object 10 do not relatively move.

In FIG. 1, the inspection object 10 has a plate shape. However, the shape of the inspection object 10 is freely selected. For example, the inspection object 10 may have a polyhedral shape or a shape having a curved surface such as a spherical shape or a columnar shape.

In some practical cases, the inspection object 10 has, for example, a hole, a notch, a projection, or a step.

Examples of the kinds of finishes of the surface of the inspection object 10 include no processing, mirror finish processing, semi-mirror finish processing, and texturing processing.

The surface inspection apparatus 1 inspects defects and the texture of the surface of the inspection object 10.

Examples of the defects include a sink mark and a weld line. The sink mark is a surface depression that is formed in a thick portion or a rib portion. The weld line is a stripe that is formed on a portion to which the end of melted resin joins in a mold. A dent and a scratch that are formed when an object hits the surface are also included in the examples of the defects.

The texture represents visual or tactile impression and is affected by the color, gloss, and unevenness of the surface of the object. The unevenness of the surface includes a stripe that is formed when a mold is cut. This kind of stripe differs from the defects.

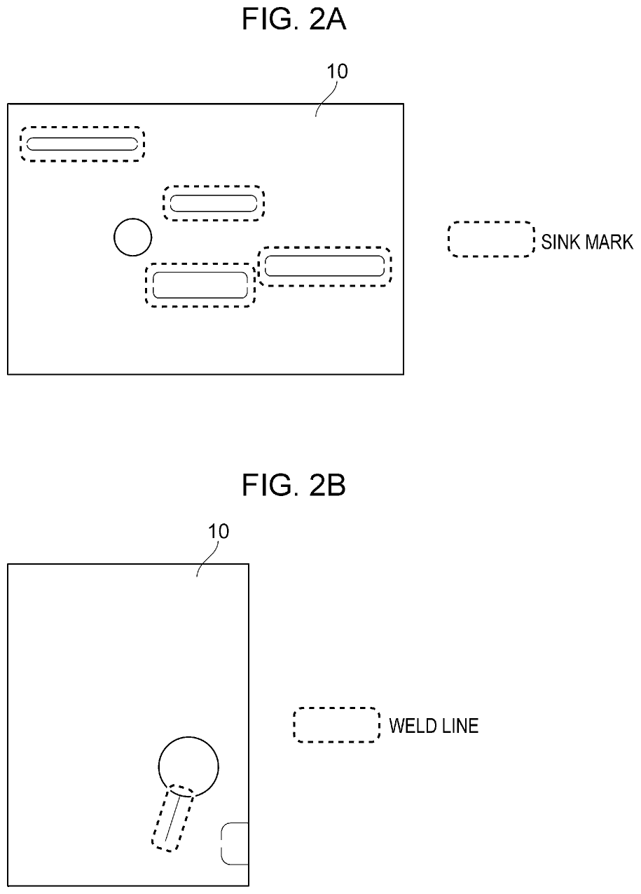

FIG. 2A and FIG. 2B illustrate examples of the defects that appear on the surface of the inspection object 10. FIG. 2A illustrates sink marks by way of example. FIG. 2B illustrates a weld line by way of example. In FIG. 2A and FIG. 2B, the defects are surrounded by lines. In FIG. 2A, there are four sink marks.

The surface inspection apparatus 1 according to the present exemplary embodiment is used not only for an inspection of the defects and the texture but also for an inspection of a stain on the surface.

The surface inspection apparatus 1 generates an image that emphasizes each defect of the surface of the inspection object 10 and quantifies and outputs the result of evaluation of the texture. The defects described herein correspond to unevenness and a stripe that appear at a portion that is originally flat, that is, the sink marks and the weld line. The texture is evaluated by a numeral. The inspection object 10 illustrated in FIG. 1 is placed so as to be parallel to a plane that is defined by the X-axis and the Y-axis. In this case, the normal of the surface of the inspection object 10 is parallel to the Z-axis.

The surface inspection apparatus 1 is disposed above the inspection object 10 in the vertical direction. In other words, the optical axis of an optical system that is used by the surface inspection apparatus 1 for imaging the inspection object 10 is set to be substantially parallel to the normal of the surface of the inspection object 10. In the following description, conditions for the optical axis are also referred to as “imaging conditions”.

At this time, the surface inspection apparatus 1 is placed at a position that satisfies the imaging conditions. The surface inspection apparatus 1 may be placed so as to be secured to a specific member or so as to detachable from the specific member.

The surface inspection apparatus 1 may be carried by an operator. In this case, the operator holds the surface inspection apparatus 1 by, for example, the hands and directs a light-receiving surface at the inspection object 10 to inspect a freely selected surface.

### Configuration of Surface Inspection Apparatus

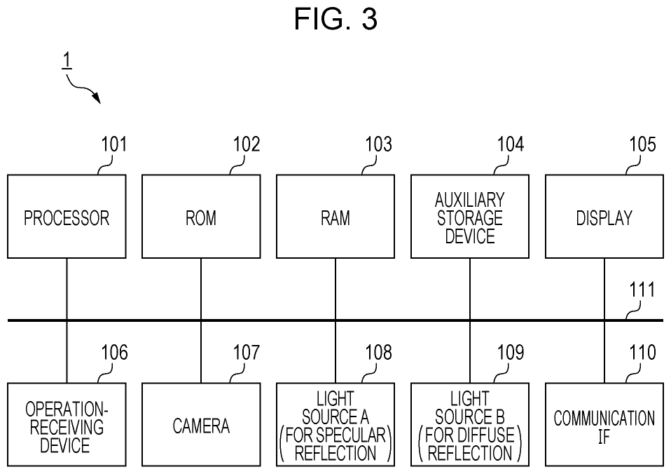

FIG. 3 illustrates an example of the hardware configuration of the surface inspection apparatus 1 that is used according to the first exemplary embodiment.

The surface inspection apparatus 1 illustrated in FIG. 3 includes a processor 101 that controls the operation of the entire apparatus, a read only memory (ROM) 102 that stores, for example, a basic input output system (BIOS), a random access memory 103 (RAM) that is used as a work area for the processor 101, an auxiliary storage device 104 that stores a program and image data, a display 105 that displays an image that is acquired by imaging the surface of the inspection object 10 and information about an operation, an operation-receiving device 106 that receives an operation from the operator, the camera 107 that images the surface of the inspection object 10, the light sources 108 and 109 that illuminate the surface of the inspection object 10, and a communication interface (IF) 110 that is used for communication with the outside. The components of the processor 101 are connected to each other via a signal line 111 such as a bus.

The processor 101, the ROM 102, and the RAM 103 function as a computer. The processor 101 achieves various functions by performing the program. For example, the processor 101 performs the program for generating an image that represents the surface of the inspection object 10 and the luminescence of illumination light.

The image data that is acquired by imaging the surface of the inspection object 10 is stored in the auxiliary storage device 104. Examples of the auxiliary storage device include a semiconductor memory and a hard disk device. The auxiliary storage device 104 also stores firmware and an application program. In the following description, the firmware and the application program are collectively referred to as a “program”.

Examples of the display 105 include a liquid-crystal display and an organic EL display, and the display 105 displays, for example, an image of the entire inspection object 10 or a specific portion of the inspection object 10. The display 105 is also used for position adjustment between the inspection object 10 and the imaging range.

According to the present exemplary embodiment, the display 105 is integrally formed with the apparatus body but may be an external device that is connected via the communication IF 110 or may be a part of another device that is connected via the communication IF 110. For example, the display 105 may be a display for another computer that is connected via the communication IF 110.

The operation-receiving device 106 is configured by using, for example, a physical switch or button that is included in the housing or a touch sensor that is included in the display 105.

A device into which the display 105 and the operation-receiving device 106 are integrally formed is called a touch screen. The touch screen is used to receive a user operation into a displayed software keyboard (also referred to as a soft keyboard).

According to the present exemplary embodiment, a color camera is used as the camera 107. A charge coupled device (CCD) imaging sensor element or a complementary metal oxide semiconductor (CMOS) imaging sensor element, for example, is used as an imaging element in the camera 107.

The use of the color camera as the camera 107 enables the luminance and the color tone of the surface of the inspection object 10 to be observed. The camera 107 is an example of an imaging device.

According to the present exemplary embodiment, white light sources are used as the light sources 108 and 109.

The light source 108 is disposed at an angle such that a light component that is reflected by specular reflection from the surface of the inspection object 10 is a principal light component that is incident on the camera 107. The light source 108 is an example of a first light source.

The light source 109 is disposed at an angle such that a light component that is reflected by diffuse reflection from the surface of the inspection object 10 is a principal light component that is incident on the camera 107. The light source 109 is an example of a second light source.

In FIG. 3, the light source 108 is denoted as a “light source A”, and the light source 109 is denoted as a “light source B”.

According to the present exemplary embodiment, the light source 108 and the light source 109 are disposed opposite each other with the optical axis of the camera 107 interposed therebetween.

According to the present exemplary embodiment, unparallel light sources are used as the light source 108 and the light source 109. That is, point light sources or surface light sources are used as the light source 108 and the light source 109.

In the surface inspection apparatus 1 according to the present exemplary embodiment, the output axis of illumination light that is emitted from the light source 108, the output axis of illumination light that is emitted from the light source 109, and the optical axis of the camera 107 are substantially on the same plane.

The communication IF 110 is configured by using a module that conforms to a wired or wireless communication standard. An Ethernet (registered trademark) module, a universal serial bus (USB), or a wireless LAN, for example, is used as the communication IF 110.

### Structure of Optical System

FIG. 4 illustrate an example of the structure of the optical system in the surface inspection apparatus 1 according to the first exemplary embodiment.

In FIG. 4, a sectional shape of the light-shielding frame 100 is schematically illustrated. However, the sectional shape illustrated in FIG. 4 is an example.

According to the present exemplary embodiment, the range of an opening of the light-shielding frame 100 that is pressed against the surface of the inspection object 10 corresponds to the imaging range. However, the range of the opening of the light-shielding frame 100 that is pressed against the surface of the inspection object 10 may be wider than the imaging range.

The opening of the light-shielding frame 100 is formed such that there is no gap between the surface of the inspection object 10 and the light-shielding frame 100 that is pressed against the surface of the inspection object 10. An elastic member composed of, for example, rubber or resin that deforms when being pressed may be attached around the opening.

A jaw portion a section of which has a V-shape is disposed at the opening of the light-shielding frame 100 illustrated in FIG. 4. The positional relationship of the camera 107 and the incident angles of the illumination light on the surface of the inspection object 10 is accurately adjusted in accordance with designed relationship by merely pressing the jaw portion against the surface of the inspection object 10.

In FIG. 4, the normal of the surface to be inspected, of the surfaces of the inspection object 10 that has a flat plate shape, is illustrated as N0, and the optical axis of the camera 107 is illustrated as L1.

In FIG. 4, the optical axis L1 is parallel to the normal N0. Specifically, the camera 107 is disposed substantially right above the inspection object 10 that has the flat plate shape.

In this case, the modulation transfer function (MTF) of the camera 107 within the field of vision is substantially uniform. For this reason, a variation in contrast due to a difference in a position within the field of vision is small, and the state of the surface of the inspection object 10 is faithfully imaged.

However, the optical axis L1 may not be strictly parallel to the normal N0, for example, provided that the optical axis L1 is substantially within 10° with respect to the normal N0.

In FIG. 4, the inspection object 10 has a substantially flat plate shape. For this reason, within the imaging range, the normal N0 at each position is substantially parallel to that at another position. Consequently, the normal N0 of the surface of the inspection object 10 is identified as a single normal.

However, the surface of the inspection object 10 practically has structural or design unevenness, a curved portion, a step, a seam, fine unevenness that is formed during, for example, molding, or another unevenness.

Accordingly, the direction in which the camera 107 is disposed is determined by using the average value of the normal N0 in a region AR to which attention is paid in the inspection object 10 or the normal N0 at a specific position P to which attention is paid. Other than these, the normal N0 of a representative portion or an average, virtual surface of the inspection object 10 may be used.

A non-telecentric lens is used as the lens of the camera 107 that is used according to the present exemplary embodiment. The unparallel light sources are used as the light sources 108 and 109 as described above.

For this reason, the size and costs of the camera 107 are smaller than those in the case where a telecentric lens and parallel light sources, for example, are used.

In FIG. 4, an angle θ_{A} formed between the output axis LA of the illumination light that is emitted from the light source 108 and the optical axis L1 of the camera 107 is set to be substantially 5°. In other words, an angle formed between the principal ray that is radiated to the surface of the inspection object 10 and the normal N0 of the surface is set to be substantially 5°.

In the case where the light source 108 is the point light source or the surface light source, the output axis LA of the illumination light means the central axis of luminous flux that is emitted from the light source 108, and this results in a direction in which luminous intensity is the maximum. The same is true for the output axis LB in the light source 109.

In the case where the angle θ_{A} is set to be less than substantially 5°, the light source 108 is likely to inhibit the light component that is reflected by the specular reflection from the surface of the inspection object 10 from being incident on the camera 107. According to the present exemplary embodiment, for this reason, the minimum of the angle θ_{A} is set to be substantially 5°.

According to the present exemplary embodiment, the maximum of the angle θ_{A} is set to be substantially 15°. However, 15° is a guide, and the maximum of the angle may be 15° or more. For example, the maximum of the angle θ_{A} may be set to be substantially 15° or more by using the telecentric lens and the parallel light sources.

In the case where the angle θ_{A} is more than substantially 15° and is more than a threshold angle, however, the principal light component that is included in reflection light that is incident on the camera 107 is changed from the light component that is reflected by the specular reflection into the light component that is reflected by the diffuse reflection.

In view of this, in an example according to the present exemplary embodiment, the percentage of the light component that is reflected by the specular reflection in the reflection light that is incident on the camera 107 is increased, the light source 108 is located so as not to inhibit the light component from being incident on the camera 107, and the angle θ_{A} of the light source 108 is set to be substantially 5°.

Consequently, the light component that is included in the illumination light that is emitted from the light source 108 and that is reflected by the specular reflection is the principal light component that is incident on the camera 107.

A phrase such as the “light component that is reflected by the specular reflection is the principal light component that is incident on the camera 107” is described herein because there is a possibility that the light component that is reflected by the diffuse reflection from the surface of the inspection object 10 is somewhat incident on the camera 107 depending on a relationship between slopes of the fine unevenness that is formed on the surface or the structural unevenness of the inspection object 10 and the angle of the illumination light.

According to the present exemplary embodiment, the illumination light that is emitted from the light source 108 is used to acquire information about the texture of the surface of the inspection object 10, particularly the gloss. This is also because a person readily notices a defect of a portion having the gloss.

The illumination light that is emitted from the light source 108 is incident on the unevenness of the surface of the inspection object 10 in the substantially vertical direction. For this reason, an image that the camera 107 acquires by imaging by using the illumination light that is emitted from the light source 108 has little information about shadow due to the unevenness of the surface of the inspection object 10.

In FIG. 4, the angle θ_{B} formed between the output axis LB of the illumination light that is emitted from the light source 109 and the optical axis L1 of the camera 107 is set to be substantially 45°.

In the case where the angle θ_{B} is set to be substantially 45°, the light component that is reflected by the diffuse reflection from the surface of the inspection object 10 is the principal light component that is incident on the camera 107.

Also, in this case, there is a possibility that the light component that is included in the illumination light that is emitted from the light source 109 and that is reflected by the specular reflection is incident on the camera 107 depending on a relationship between the structural or design unevenness of the inspection object 10 or the like and the angle of the illumination light. For this reason, a phrase such as the “light component that is reflected by the diffuse reflection from the surface of the inspection object 10 is the principal light component that is incident on the camera 107” is used.

According to the present exemplary embodiment, the light source 108 and the light source 109 are disposed opposite each other with the optical axis L1 of the camera 107 interposed therebetween. For this reason, directions in which shadow that is formed in association with the unevenness of the surface of the inspection object 10 extends are opposite each other in principle.

In other words, the direction of the shadow that is formed by the illumination light that is emitted from the light source 108 in association with the unevenness of the surface of the inspection object 10 is opposite the direction of the shadow that is formed by the illumination light that is emitted from the light source 109 in association with the same unevenness of the surface of the inspection object 10.

The unevenness of the surface of the inspection object 10 is illuminated with the illumination light that is emitted from the light source 109 diagonally from above. Accordingly, the shadow of a projecting portion on the surface appears so as to be away from the light source 109, and the shadow of a depressed portion on the surface appears so as to approach the light source 109.

Accordingly, in the case where the surface of the inspection object 10 is imaged from above in the vertical direction, it may be made easy to observe the unevenness of the surface of the inspection object 10 by using the light source 108 and the light source 109.

### Inspection Operation

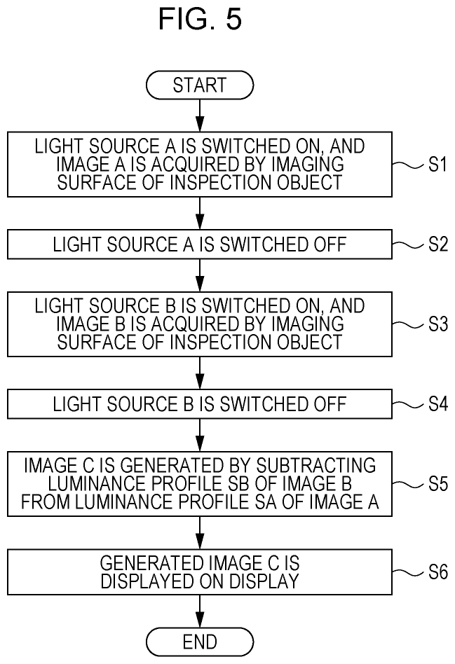

FIG. 5 is a flowchart illustrating an example of the inspection operation of the surface inspection apparatus 1. Symbols S illustrated in the figure mean steps.

The surface inspection apparatus 1 according to the present exemplary embodiment starts the inspection operation, switches the light source A on, and acquires an image A by imaging the surface of the inspection object 10 (step 1). The light source A described herein is the light source 108. The image A is an example of a first image.

When imaging for the image A ends, the surface inspection apparatus 1 switches the light source A off (step 2).

Subsequently, the surface inspection apparatus 1 switches the light source B on and acquires an image B by imaging the surface of the inspection object 10 (step 3). The light source B described herein is the light source 109. The image B is an example of a second image.

When imaging for the image B ends, the surface inspection apparatus 1 switches the light source B off (step 4).

Subsequently, the surface inspection apparatus 1 generates an image C by subtracting a luminance profile SB of the image B from a luminance profile SA of the image A (step 5) and displays the generated image C on the display 105 (see FIG. 3) (step 6).

The luminance profiles SA and SB described herein represent so-called intensity value distribution of a luminance signal. The image C is an example of a third image and contains information about the texture such as the gloss and so on.

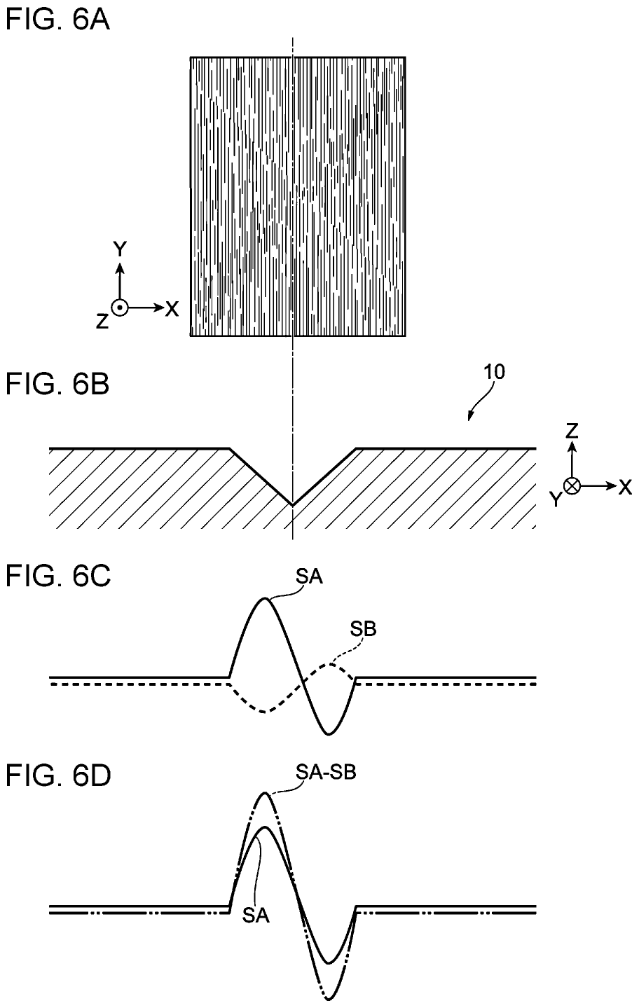

FIG. 6A, FIG. 6B, FIG. 6C, and FIG. 6D illustrate the principle of the inspection of the surface inspection apparatus 1 according to the first exemplary embodiment. FIG. 6A illustrates the image C by way of example. FIG. 6B illustrates a section of a depressed defect that is formed on the surface of the inspection object 10. FIG. 6C illustrates the luminance profile SA of the image A and the luminance profile SB of the image B. FIG. D illustrates a luminance profile SA-SB related to the image C and the luminance profile SA of the image A.

In FIG. 6B, a depression is formed on the surface of the inspection object 10. The depression is an example of the sink mark. The depression illustrated in FIG. 6B has a sectional shape of an isosceles triangle for convenience of description. However, the shape is an example.

In this case, as for the luminance profile SA illustrated in FIG. 6C, the intensity of a light component that is reflected from a left-hand slope viewed in front of the paper is larger than the intensity of a light component that is reflected from a right-hand slope. The distribution of the intensity value occurs because the light source 108 is on the right of the depression. A principal light component that is contained in the luminance profile SA is the light component that is reflected by the specular reflection.

As for the luminance profile SB illustrated in FIG. 6C, however, the intensity of a light component that is reflected from the left-hand slope viewed in front of the paper is smaller than the intensity of a light component that is reflected from the right-hand slope.

The distribution of the intensity value occurs because the light source 109 is on the left of the depression. A principal light component that is contained in the luminance profile SB is the light component that is reflected by the diffuse reflection.

FIG. 6D illustrates the luminance profile SA-SB related to the image C. For comparison, FIG. 6D also illustrates the luminance profile SA related to the image A.

As illustrated in FIG. 6D, the amplitude of the luminance profile SA-SB for the image C is larger by the luminance profile SB related to the diffuse reflection than that in the case of using only the luminance profile SA.

Consequently, as for the image C illustrated in FIG. 6A, the contrast of the defect is emphasized. As a result of the emphasized contrast, the unevenness is likely to be conspicuous.

The image C contains shadow due to the illumination light that is radiated from both regions with the optical axis Ll interposed therebetween. The image A that is acquired by using the illumination light from the light source 108 has little information about the shadow.

The camera 107 of the surface inspection apparatus 1 that is used according to the present exemplary embodiment is the non-telecentric lens.

The surface inspection apparatus 1 calculates a difference between the light component that is reflected by the specular reflection and the light component that is reflected by the diffuse reflection, that is, generates the image C related to the luminance profile SA-SB.

### Second Exemplary Embodiment

In an example described according to the present exemplary embodiment, the image C is displayed on the display 105 (see FIG. 3) with an indicator that specifies a location to be inspected superposed thereon.

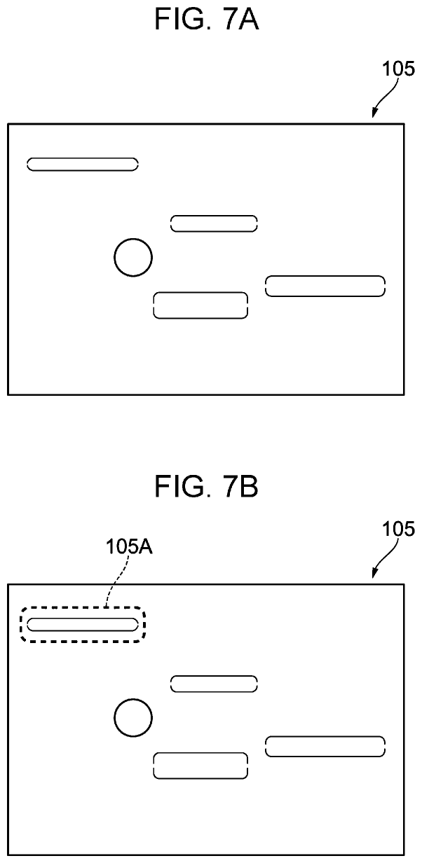

FIG. 7A and FIG. 7B illustrate examples of the image C that is displayed according to a second exemplary embodiment. FIG. 7A illustrates, by way of example, the image C that is acquired by imaging the inspection object 10 (see FIG. 1) and that is displayed on the display 105 as it is. FIG. 7B illustrates, by way of example, the image C that is acquired by imaging the inspection object 10 and that is displayed with the indicator superposed thereon.

The example of the display illustrated in FIG. 7A corresponds to the image C that is displayed on the display 105 of the surface inspection apparatus 1 (See FIG. 1) that is used according to the first exemplary embodiment. In this case, the operator uses the image C that emphasizes each defect to determine an anomaly in the texture and the defect.

If the determination completely depends on the operator, a location to be checked is overlooked in some cases.

As for the example of the display illustrated in FIG. 7B, a frame 105A is displayed at a location to be checked on the inspection object 10 to prevent the location to be checked from being overlooked.

The frame 105A illustrated in FIG. 7B is generated by the processor 101 (See FIG. 3) depending on, for example, the shape or size of the defect that appears at the location.

In FIG. 7B, the frame 105A is displayed as the indicator near the upper left corner. However, the frame 105A may be displayed at a different position. The position at which the frame 105A is displayed may be changed in order for every single location per predetermined cycle.

The number of the frame 105A that is displayed at the same time may be plural. For example, the frames 105A may be displayed at all of locations that are surrounded by dashed lines in FIG. 2A and FIG. 2B.

The location to be checked may be inhibited from being overlooked regardless of the proficiency of the operator by displaying the frame 105A.

The form of displayed blinking, the kind of a line, the thickness of the line, and the color of the displayed frame 105A, for example, may be determined depending on the environment of the inspection or the inspection object 10. For example, an opposite color or a complementary color of the color tone of the inspection object 10 may be used as the color of the frame 105A to improve the visibility of the location to be checked.

Various methods of determining the location at which the frame 105A is displayed are thought.

For example, in the case where the position of the surface inspection apparatus 1 and the position of the inspection object 10 are uniquely determined, the position on the screen at which the frame 105A is displayed is set in advance. In other words, in the case where the position of the inspection object 10 is adjusted to be the position that is set in advance for the surface inspection apparatus 1, the position on the screen at which the frame 105A is displayed or the shape thereof, for example, is set in advance.

In the case where the position of the surface inspection apparatus 1 and the position of the inspection object 10 are not uniquely determined, however, a structurally characteristic point that is contained in the image C that is acquired by imaging is used as a criterion, and the processor 101 (See FIG. 3) sets the position at which the frame 105A is displayed.

### Third Exemplary Embodiment

Also, in an example described according to the present exemplary embodiment, the image C is displayed on the display 105 (see FIG. 3) with the indicator that specifies the location to be inspected superposed thereon.

According to the present exemplary embodiment, however, the indicator is physically attached to the surface inspection apparatus 1.

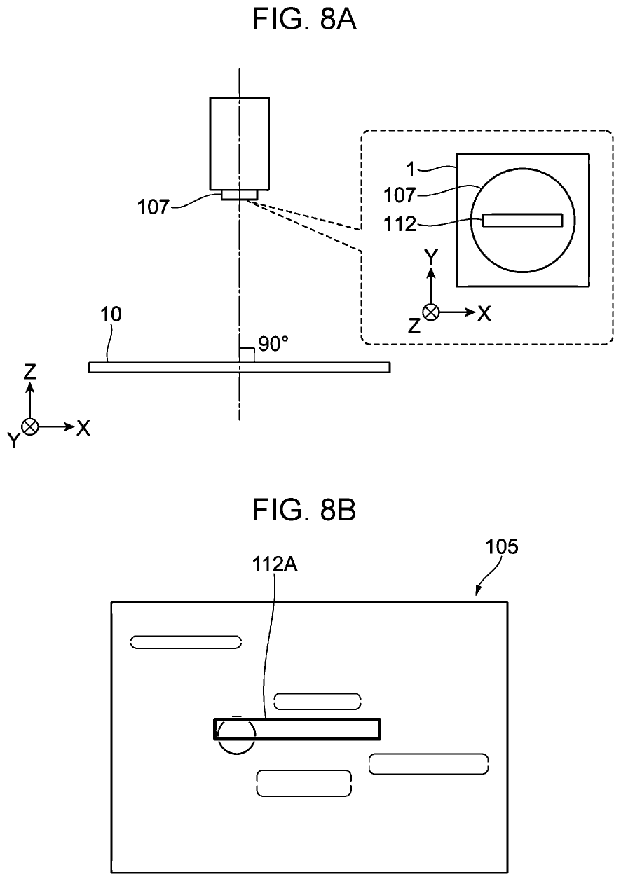

FIG. 8A and FIG. 8B illustrate examples of the image C that is displayed according to a third exemplary embodiment. FIG. 8A illustrates a position at which an indicator 112 is attached. FIG. 8B illustrates an example of the image C that is acquired by imaging the inspection object 10 and that is displayed.

According to the present exemplary embodiment, as illustrated in FIG. 8A, the indicator 112 is physically placed on the light-receiving surface of the camera 107. Specifically, the indicator 112 is placed between the light-receiving surface and the inspection object 10.

For this reason, as illustrated in FIG. 8B, the display 105 displays a frame 112A corresponding to the indicator 112.

It may be facilitated to make position adjustment between the surface inspection apparatus 1 that is stationary and the inspection object 10 and position adjustment between the inspection object 10 that is stationary and the surface inspection apparatus 1 by displaying the frame 112A on the display 105.

### Fourth Exemplary Embodiment

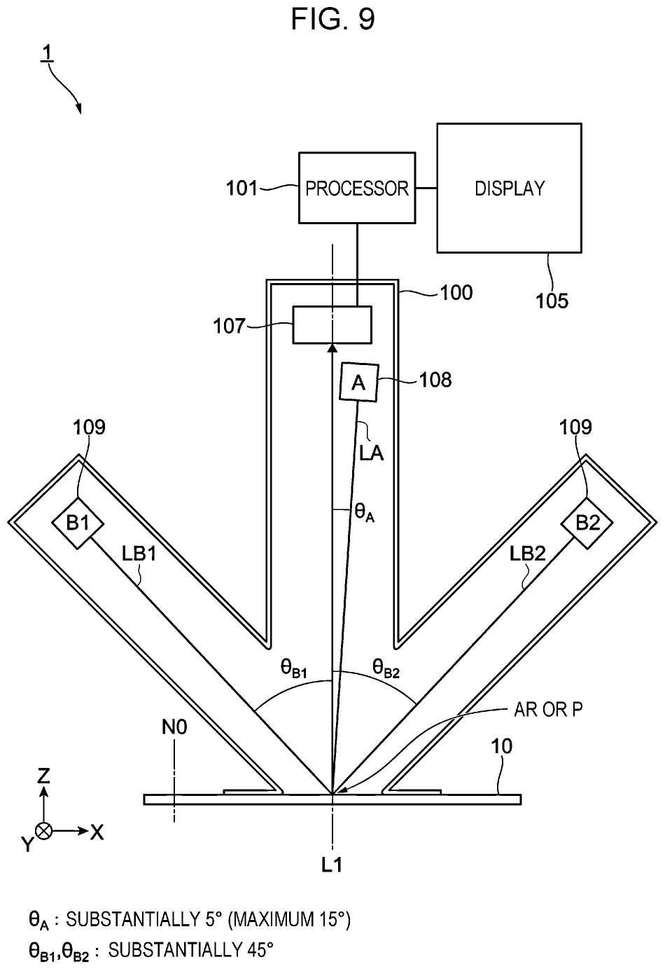

FIG. 9 illustrates the arrangement of an optical system in a surface inspection apparatus 1 according to a fourth exemplary embodiment. In FIG. 9, components corresponding to those in FIG. 4 are designated by like reference characters.

The surface inspection apparatus 1 illustrated in FIG. 9 includes two light sources 109.

One of the two light sources 109 is disposed at the same position as the light source 109 according to the first exemplary embodiment. In FIG. 9, the light source 109 is denoted as “B1”, and the output axis thereof is denoted as “LB1”. An angle between the output axis LB1 and the optical axis L1 of the camera 107 is denoted as θ_{B1}. An image that is acquired by imaging the reflection light related to the light source B1 is denoted as “B1”.

The other light source 109 that is added in FIG. 9 is disposed in the same region as the light source 108. In FIG. 9, the added light source 109 is denoted as “B2”, and the output axis thereof is denoted as “LB2”. An angle between the output axis LB2 and the optical axis L1 of the camera 107 is denoted as θ_{B2}. An image that is acquired by imaging the reflection light related to the light source B2 is denoted as “B2”. The light source B2 is an example of a second light source.

According to the present exemplary embodiment, the angle θ_{B1} and the angle θ_{B2} are substantially equal to each other.

However, the angle θ_{B1} and the angle θ_{B2} may differ from each other. The angle θ_{B1} and the angle θ_{B2} are set to be within the range in which the light component that is reflected by the diffuse reflection from the surface of the inspection object 10 is the principal light component that is incident on the camera 107.

According to the present exemplary embodiment, the output axis LA of the illumination light that is emitted from the light source 108, the output axis LB1 of the illumination light that is emitted from the light source B1, the output axis LB2 of the illumination light that is emitted from the light source B2, and the optical axis L1 of the camera 107 are substantially on the same plane.

The angle θ_{B2} of the light source B2 is larger than the angle θ_{A} of the light source A. Accordingly, the length of shadow that appears in the image B2 is longer than that length of shadow that appears in the image A even when the same unevenness is imaged.

In the case where the light source B1 and the light source B2 are disposed opposite each other with the optical axis L1 of the camera 107 interposed therebetween, directions in which the shadow extends are opposite each other between two images B that are acquired by imaging the illumination light of the light sources.

### Fifth Exemplary Embodiment

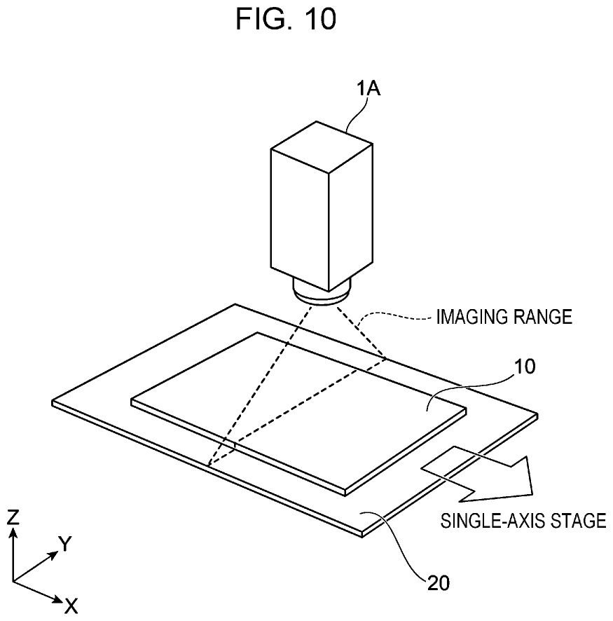

FIG. 10 illustrates an example of the use of a surface inspection apparatus 1A that is supposed according to a fifth exemplary embodiment. In FIG. 10, a component corresponding to that in FIG. 1 is designated by a like reference character.

The surface inspection apparatus 1A that is used according to the present exemplary embodiment uses a so-called line camera. For this reason, the imaging range is linear.

According to the present exemplary embodiment, the inspection object 10 is moved in the direction of an arrow with the inspection object 10 placed on a single-axis stage 20 during inspection. The entire inspection object 10 is imaged by moving the single-axis stage 20 in one direction.

A relationship in arrangement of the camera 107, the light source 108 (See FIG. 3), and the light source 109 (See FIG. 3) is the same as that according to the first exemplary embodiment except that a line camera is used as the camera 107 (See FIG. 3).

Specifically, it may be thought that the light-receiving surface of the camera 107 linearly extends in the Y-axis, that is, in the direction toward the back of the paper in FIG. 4.

### Other Exemplary Embodiments

(1) The exemplary embodiments of the present disclosure are described above. However, the technical range of the exemplary embodiments of the present disclosure is not limited by the range described according to the exemplary embodiments described above. It is clear from the recitation of claims that exemplary embodiments that are acquired by modifying or altering the exemplary embodiments described above in various ways are in the technical range of the exemplary embodiments of the present disclosure.

(2) According to the exemplary embodiments described above, the color camera is used as the camera 107 (See FIG. 3). However, a monochrome camera may be used. The surface of the inspection object 10 (See FIG. 1) may be inspected by using only a green (G) component of the color camera.

(3) According to the exemplary embodiments described above, the white light sources are used as the light sources 108 and 109 (See FIG. 3). However, the color of the illumination light may be freely selected.

The illumination light is not limited to the visible light but may be infrared light, ultraviolet light, or another light. When the infrared light or the ultraviolet light, for example, is used as the illumination light, the positions at which the light source 108 and the light source 109 are disposed are defined by using a relationship between the specular reflection and the diffuse reflection.

(4) According to the exemplary embodiments described above, the maximum of the angle θ_{A} is substantially 15°. However, in the case where the telecentric lens that makes the principal ray parallel to the optical axis of the lens is used as the camera 107, the maximum of the angle θ_{A} may be substantially 25°.

(5) According to the exemplary embodiments described above, the angle θ_{B} is substantially 45° but may be in the range from substantially 35° to substantially 55°.

(6) According to the exemplary embodiments described above, the image C is generated by subtracting the image B that is acquired by imaging the reflection light of the light source 109 from the image A that is acquired by imaging the reflection light of the light source 108. However, the images may be separately displayed on the display 105.

(7) According to the exemplary embodiments described above, the images are captured by switching the light source 108 and the light source 109 on and off. However, the images may be captured with the light source 108 and the light source 109 simultaneously switched on.

(8) In the description according to the above exemplary embodiments, the output axis LA of the illumination light that is emitted from the light source 108, the output axis LB of the illumination light that is emitted from the light source 109, and the optical axis L1 of the camera 107, for example, are substantially on the same plane. However, the light source 108 or the light source 109 may be on a different plane.

(9) In the embodiments above, the term “processor” refers to hardware in a broad sense. Examples of the processor include general processors (e.g., CPU: Central Processing Unit) and dedicated processors (e.g., GPU: Graphics Processing Unit, ASIC: Application Specific Integrated Circuit, FPGA: Field Programmable Gate Array, and programmable logic device).

In the embodiments above, the term “processor” is broad enough to encompass one processor or plural processors in collaboration which are located physically apart from each other but may work cooperatively. The order of operations of the processor is not limited to one described in the embodiments above, and may be changed.

The foregoing description of the exemplary embodiments of the present disclosure has been provided for the purposes of illustration and description. It is not intended to be exhaustive or to limit the disclosure to the precise forms disclosed. Obviously, many modifications and variations will be apparent to practitioners skilled in the art. The embodiments were chosen and described in order to best explain the principles of the disclosure and its practical applications, thereby enabling others skilled in the art to understand the disclosure for various embodiments and with the various modifications as are suited to the particular use contemplated. It is intended that the scope of the disclosure be defined by the following claims and their equivalents.

What is claimed is:

1. A surface inspection apparatus comprising:
an imaging device that images a portion of an object to be inspected;
a first light source that is included in a plurality of light sources that illuminate the portion, the first light source being configured such that a light component that is included in light emitted from the first light source and that is reflected by specular reflection from the portion to be inspected is a principal light component that is incident on the imaging device; and
a second light source that is included in the plurality of light sources and that is disposed opposite the first light source with an optical axis of the imaging device interposed therebetween such that a light component that is reflected by diffuse reflection from the portion to be inspected is a principal light component that is incident on the imaging device.

2. The surface inspection apparatus according to claim 1,
wherein the optical axis of the imaging device is substantially parallel to a normal of the portion.

3. The surface inspection apparatus according to claim 2,
wherein a slope of the optical axis with respect to the normal is substantially within 10°.

4. The surface inspection apparatus according to claim 3,
wherein a slope of an output axis of the first light source with respect to the optical axis is substantially from 5° to 15°.

5. The surface inspection apparatus according to claim 3,
wherein a slope of an output axis of the second light source with respect to the optical axis is substantially 45°.

6. The surface inspection apparatus according to claim 4,
wherein a slope of an output axis of the second light source with respect to the optical axis is substantially 45°.

7. The surface inspection apparatus according to claim 1,
wherein the imaging device, the first light source, and the second light source are substantially on the same plane.

8. The surface inspection apparatus according to claim 2,
wherein the imaging device, the first light source, and the second light source are substantially on the same plane.

9. The surface inspection apparatus according to claim 3,
wherein the imaging device, the first light source, and the second light source are substantially on the same plane.

10. The surface inspection apparatus according to claim 4,
wherein the imaging device, the first light source, and the second light source are substantially on the same plane.

11. The surface inspection apparatus according to claim 5,
wherein the imaging device, the first light source, and the second light source are substantially on the same plane.

12. The surface inspection apparatus according to claim 6,
wherein the imaging device, the first light source, and the second light source are substantially on the same plane.

13. The surface inspection apparatus according to claim 1,
wherein an image that the imaging device acquires by imaging contains an image of an indicator representing an inspection range.

14. The surface inspection apparatus according to claim 13,
wherein the image of the indicator is combined by image processing with the image that the imaging device acquires by imaging.

15. The surface inspection apparatus according to claim 13,
wherein the image of the indicator is acquired by imaging an indicator that is physically disposed on the optical axis.

16. The surface inspection apparatus according to claim 1,
wherein the first light source and the second light source emit visible light.

17. The surface inspection apparatus according to claim 16,
wherein the visible light is white.

18. The surface inspection apparatus according to claim 1,
wherein the imaging device outputs a luminance signal.

19. The surface inspection apparatus according to claim 1, further comprising:
a processor configured to:
output a third image by subtracting a luminance profile of a second image that is acquired by imaging by using the second light source from a luminance profile of a first image that is acquired by imaging by using the first light source.

20. A surface inspection apparatus comprising:
imaging means for imaging a portion of an object to be inspected;
first illuminating means for illuminating the portion, the first illuminating means being configured such that a light component that is included in light emitted from the first illuminating means and that is reflected by specular reflection from the portion to be inspected is a principal light component that is incident on the imaging device; and
second illuminating means for illuminating the portion, the second illuminating means being disposed opposite the first illuminating means with an optical axis of the imaging device interposed therebetween such that a light component that is reflected by diffuse reflection from the portion to be inspected is a principal light component that is incident on the imaging device.
